(19) United States
(12) Patent Application Publication
(10) Pub. No.: US 2002/0188082 A1
(43) Pub. Date: Dec. 12, 2002

(54) Novel vulcanizing agent for diene-based rubber and rubber composition using the same

(75) Inventors: Yuko Nakata, Hiratsuka-shi (JP); Kazunori Ishikawa, Hiratsuka-shi (JP)

Correspondence Address: ARENT FOX KINTNER PLOTKIN & KAHN, PLLC, Suite 600, 1050 Connecticut Avenue, N.W., Washington, DE, 20036-5339, US

(73) Assignee: THE YOKOHAMA RUBBER CO, LTD.

(21) Appl. No.: 09/797,679

(22) Filed: Mar. 5, 2001

(30) Foreign Application Priority Data
Mar. 23, 2000 (JP) 2000-87131
Oct. 18, 2000 (JP) 2000-318360

(51) Int. Cl. C07C 321/00; C08F 8/34; C08F 136/20

(52) U.S. Cl. 525/332.4; 568/21; 525/332.6

(57) ABSTRACT

A novel vulcanizing agent giving a vulcanized rubber superior in heat aging resistance and reversion resistance, comprising an organic sulfur vulcanizing agent obtained by reaction of sulfur and norbornene or its derivative or dicyclopentadiene in the presence of catalyzing amounts of a vulcanization accelerator and an accelerator activator.

### BACKGROUND OF THE INVENTION

1. Field of the Invention

The present invention relates to a novel vulcanizing agent of an organic sulfur compound, more particularly relates to a vulcanizing agent giving a vulcanized rubber which has a high modulus and is superior in heat aging resistance and reversion resistance without inviting a decline in the general rubber physical properties.

2. Description of the Related Art

In the past, sulfur has been mainly used as a vulcanizing agent for vulcanization of rubber. It is known, however, that the cross-linking of rubber vulcanized by sulfur consists of polysulfide bonds. These polysulfide bonds become thermally unstable along with an increase of the number of sulfur atoms, so there is a problem in the heat resistance. To deal with this problem, the method of using as a vulcanizing agent a polysulfide polymer suppressing the number of sulfur atoms of the polysulfide bonds to the extent of tetrasulfide bonds has been proposed, but this is still short of the mark in terms of the heat buildup etc. On the other hand, as a vulcanizing agent of an organic sulfur compound, there is known the reaction product of sulfur and an olefin (Japanese Unexamined Patent Publication (Kokai) No. 7-309977). In the process of production thereof, the weight of the olefin with respect to the sulfur was a low one of less than 30 wt % and a basic catalyst was used as the catalyst for the reaction. Most of these are proposed for improving the dispersion of the sulfur in the rubber (for resistance to blooming). The content of sulfur is large and the number of sulfur atoms of the polysulfide bonds in the product is an average 6.5 or so.

### SUMMARY OF THE INVENTION

The present invention takes note of the fact that the number of sulfur atoms in the polysulfide polymer for vulcanization of rubber is reflected as it is in the cross-linking of the vulcanized rubber, that is, the number of sulfur atoms of the polysulfide bonds, and has as its object to synthesize an organic sulfur compound having a number of sulfur atoms of an extent of tetrasulfide bonds and to provide this as a novel vulcanizing agent.

According to a first aspect of the present invention, there is provided an organic sulfur vulcanizing agent of the following formula 1 obtained by a reaction between sulfur and norbornene or its derivative or dicyclopentadiene in the presence of catalyzing amounts of a vulcanization accelerator and an accelerator activator:

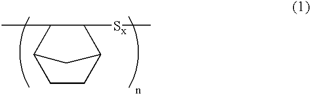

wherein, x is an integer of 1 to 10 and n is an integer of 1 to 200.

Preferably, the sulfur is contained in an amount of 30 to 70 wt %.

Further, in the present invention, there is provided an organic sulfur vulcanizing agent which is a compound expressed by the following formula 2 or 3:

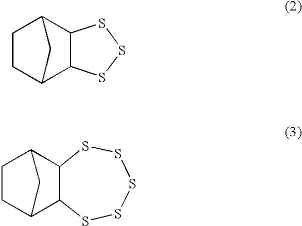

According to a second aspect of the present invention, there is provided a rubber composition comprised of 0.5 to 10 parts by weight of an organic sulfur vulcanizing agent as set forth in any one of claims 1 to 5 or 0.5 to 10 parts by weight of said organic sulfur vulcanizing agent and 0 to 10 parts by weight of sulfur with respect to 100 parts by weight of a diene-based rubber.

### DESCRIPTION OF THE PREFERRED EMBODIMENTS

The present inventors discovered that by using as a vulcanizing agent an organic sulfur compound obtained by reacting predetermined amounts of sulfur and norbornene or its derivative or dicyclopentadiene at 100 to 130° C. while stirring in the presence of catalyzing amounts of a vulcanization accelerator and an accelerator activator for a rubber composition instead of a conventional sulfur vulcanizing agent, it is possible to obtain a vulcanized rubber superior in heat aging resistance and reversion resistance without inviting a reduction in the breaking strength, the elongation at break, and other general rubber physical properties.

As the catalyst used for the synthesis of the vulcanizing agent of the present invention, an amine or a catalyst used as a general rubber vulcanization accelerator and an accelerator activator in the past may be used. For example, as the vulcanization accelerator, sulfenamide-based, guanidine-based, thiazole-based, and thiuram-based vulcanization accelerators may be mentioned, while as the accelerator activator, active zinc white having a particle size of 0.05 to 0.2 μm or zinc white having a particle size of 0.3 to 1 μm may be mentioned. Further, as the catalyzing amounts of these catalysts, the amount of 0.1 to 5 wt % with respect to the norbornene or its derivative or dicyclopentadiene is normally used.

As the norbornene or its derivative used in the present invention, norbornene, 5-norbornene-2-carboxylic acid methylester, 5-norbornene-2-carboxylic acid butylester, 5-norbornene-2-carboxylic acid octylester, 5-norbornene-2-carboxylic acid octadecylester, 5-norbornene-2-methylol, 5-norbornene-2-dimethylol, 5-norbornene-2,3-dicarboxylic acid anhydride, etc. may be mentioned. In particular, a vulcanizing agent obtained from norbornene is preferable in terms of the physical properties of the vulcanized rubber.

The sulfur-norbornene (NB) reaction product used in the present invention is obtained by reacting 3 moles of sulfur with 1 mole of norbornene to synthesize the vulcanizing agent of the above formula (2). Further, a vulcanizing agent with over 3 moles of sulfur is synthesized by further reacting sulfur with the compound of the above formula (2) in the presence of a basic catalyst. The specific structure of the sulfur-norbornene reaction product with over 3 moles of sulfur is shown in the above formula (1) or (3). The thus obtained vulcanizing agent includes 30 to 70 wt % of sulfur. In particular, one with an n in the above formula (1) of an average 4 is preferable and the rubber composition using it exhibits superior heat aging resistance.

The molar ratio (sulfur/DCPD) of the sulfur to dichloropentadiene (DCPD) used for the synthesis of the vulcanizing agent of the present invention is made not more than 5/1. When the molar ratio is over 5/1, the number of sulfur atoms of the polysulfide bond present in the organic sulfur compound produced exceeds an average of 5. Therefore, the heat aging resistance of the rubber composition using this vulcanizing agent becomes inferior. The organic sulfur compound containing 30 to 70 wt % of sulfur in the molecule has the effect of giving superior heat aging resistance and reversion resistance.

In the rubber composition of the present invention, 0.5 to 10 parts by weight of an organic sulfur vulcanizing agent, preferably 1 to 5 parts by weight, or 0.5 to 10 parts by weight of an organic sulfur vulcanizing agent and 0 to 10 parts by weight of sulfur (preferably 0.3 to 2 parts by weight) were blended into 100 parts by weight of a diene-based rubber. When the amount of these vulcanizing agents is too small, the desired heat aging resistance and reversion resistance cannot be exhibited. Further, when the amount blended is too large, the rubber becomes too hard.

As the diene-based rubber ingredient used in the rubber composition of the present invention, any diene-based rubber may be used. For example, natural rubber (NR), polyisoprene rubber (IR), various styrene-butadiene copolymer rubbers (SBR), various polybutadiene rubbers (BR), acrylonitrile-butadiene copolymer rubber (NBR), butyl rubber (IIR), and other diene-based rubbers may be used alone or in any blends.

The rubber composition using the vulcanizing agent of the present invention may have further blended into it a reinforcing filler such as carbon black or silica, a vulcanizing agent or vulcanization accelerator, various oils, an antioxidant, a filler, a softening agent, a plasticizer, or other various compounding agents and additives blended into general rubber compositions. The amounts of these compounding agents and additives blended may be made the generally used amounts.

In particular, the rubber composition in which 2.0 parts by weight of a vulcanization accelerator were blended into 100 parts by weight of a diene-based rubber exhibits superior heat aging resistance. Further, the rubber composition in which 0.1-5.0 parts by weight of an aromatic carboxylate with respect to 100 parts by weight of a diene-rubber were blended together with a vulcanizing agent of the present invention exhibits an even more superior that aging resistance. As the aromatic carboxylate ingredient used in the rubber composition of the present invention, any aromatic carboxylate may be used. For example, zinc benzoate, cobalt benzoate, nickel benzoate, ammonium benzoate, and other aromatic carboxylates may be used alone or in any blends. The rubber composition using these aromatic carboxylates exhibits superior heat aging resistance and also a high modulus, a high breaking strength and a high elongation at break.

Further, in a rubber composition containing silica, a sulfur-containing silane coupling agent (for example, Si69 or Si75 made by Degussa-Huls) may be used. In this case as well, the vulcanizing agent of the present invention can be effectively used.

Below, the present invention will be explained in further detail using examples and comparative examples, but the present invention is of course not limited to the scope of these examples.

The composition of the IR (master batch) used in Table 1 is as follows:

| Ingredient | Parts by weight |
|---|---|
| IR (Nipol IR2200 made by | 100 |
| Nippon Zeon) | |
| N339 Carbon Black (Seast KH | 50 |
| made by Tokai Carbon) | |
| Zinc oxide (Zinc White No. 3) | 3 |
| Industrial use stearic acid | 1 |
| Antioxidant (N-phenyl-N′-(1,3- | 1 |
| dimethylbutyl)-P-phenylenediamine | |

Production of NB3S (NB/S=1/3 (molar ratio)) Organic Sulfur Vulcanizing Agent of Table 1

(1) Synthesis of Norbornene Trisulfide

96 g (3.0 moles) of sulfur and 94.15 g (1.0 mole) of norbornene were placed in 100 ml of a DMF solution in a reaction flask. 0.8 g each of a vulcanization accelerator (N-cyclohexyl-2-benzothiazolesulfenamide) and an accelerator activator (zinc oxide) was added as a catalyst, then the mixture was heated at 120 to 130° C. and stirred for 26 hours. After the end of the reaction, the DMF was removed in vacuo to obtain 183.4 g (96.5%) of a black liquid of norbornene trisulfide (formula 2).

Production of NB4S (NB/S=1/4 (molar ratio)) Organic Sulfur Vulcanizing Agent of Table 1

(2) Synthesis of Norbornene Tetrasulfide

1.68 g (0.053 mole) of sulfur and 10 g (0.053 mole) of norbornene trisulfide were placed in a reaction flask. 0.02 g of triethylamine (Et₃N) was added as a catalyst, then the mixture was heated at 50 to 90° C. and stirred for 6 hours. After the end of the reaction, 11.2 g (95.9%) of a black viscous substance of a mixture of a polymer, the above formula (1), NB3S (2), and NB5S (3) was obtained. The content of NB3S (2) and NB5S (3) was 52 wt %.

Production of NBPS (NB/S=1/5 (molar ratio)) Organic Sulfur Vulcanizing Agent of Table 1

(3) Synthesis of Norbornene Polysulfide

3.37 g (0.105 mole) of sulfur and 10 g (0.053 mole) of norbornene trisulfide were placed in a reaction flask. 0.02 g of triethylamine (Et₃N) was added as a catalyst, then the mixture was heated at 50 to 90° C. and stirred for 6 hours. After the end of the reaction, 12.5 g (93.5%) of a black viscous substance of a mixture of a polymer, the above formula (1), NB3S (2), and NB5S (3) was obtained. The content of NB3S (2) and NB5S (3) was 31 wt %.

Production of DCPD/S (1) (50/50 wt % ratio) Organic Sulfur Vulcanizing Agent of Table 1

60 g (1.875 mole) of sulfur and 60 g (0.455 mole) of DCPD were placed in a reaction flask. 0.02 g to 0.8 g of each of a N-cyclohexyl-2-benzotriazolesulfenamide vulcanization accelerator and a zinc oxide accelerator activator were added as a catalyst, then the mixture was heated at 100 to 130° C. and stirred for 8 hours. After the end of the reaction, 58 g of a black viscous substance of an organic sulfur compound was obtained.

Production of DCPD/S (2) (60/40 wt % ratio) Organic Sulfur Vulcanizing Agent of Table 1

60 g (1.875 mole) of sulfur and 90 g (0.683 mole) of DCPD were placed in a reaction flask. 0.02 g to 0.8 g of each of a N-cyclohexyl-2-benzotriazolesulfenamide vulcanization accelerator and a zinc oxide accelerator activator were added as a catalyst, then the mixture was heated at 100 to 130° C. and stirred for 8 hours. After the end of the reaction, 150 g of a black viscous substance of an organic sulfur compound was obtained.

### EXAMPLES 1 TO 8 AND COMPARATIVE EXAMPLE 1

In the present examples, the formulations shown in Table 1 were used to obtain the rubber compositions of the examples. These were used for tensile tests and rheometer tests.

Preparation of Test Samples

The ingredients of the IR master batch except for the vulcanization accelerator, sulfur, and vulcanizing agent of the present invention (NB3S, NB4S, NBPS, DCPD/S (1), DCPD/S (2)) were kneaded for 3 to 5 minutes in a 1.8 liter closed mixer. When the temperature reached 165±5° C., the IR master batch was discharged. The vulcanization accelerator, sulfur, and the predetermined vulcanizing agent were kneaded in this by an 8-inch open roll to obtain a rubber composition. Next, this rubber composition was vulcanized at 160° C. for 20 minutes in a 15 cm×15 cm×0.2 cm mold to prepare a test piece (rubber sheet).

Test Methods

1) Tensile strength (T_{B}), elongation (E_{B}), and 100% and 300% tensile stress (MPa): Measured in accordance with the test method of JIS K6301

2) Vulcanization test by rheometer: Measured in accordance with the test method of SRIS-3102 (Japan Society of Rubber Industry Standard)

The results are shown in the following Table 1.

TABLE 1

| | Comp. | | | | | | | | |
|---|---|---|---|---|---|---|---|---|---|
| | Ex. 1 | Ex. 1 | Ex. 2 | Ex. 3 | Ex. 4 | Ex. 5 | Ex. 6 | Ex. 7 | Ex. 8 |
| IR (master batch) | 155 | 155 | 155 | 155 | 155 | 155 | 155 | 155 | 155 |
| NB3S | — | 3.9 | — | — | — | — | — | — | — |
| NB4S | — | — | 3.1 | 1.56 | — | — | — | — | — |
| NBPS | — | — | — | — | 2.8 | — | — | — | — |
| DCPD/S(1) | — | — | — | — | — | 3.0 | 2.5 | 1.0 | — |
| DCPD/S(2) | — | — | — | — | — | — | — | 1.0 | |
| Sulfur*1 | 1.5 | — | — | 0.9 | — | — | 0.25 | 1.0 | 1.0 |
| Vulcanization | 1.00 | 2.00 | 2.00 | 1.50 | 2.00 | 2.50 | 2.25 | 1.50 | 1.50 |
| accelerator*2 | | | | | | | | | |

Vulcanization conditions: 160° C., 20 minutes; blank tension

| | | | | | | | | | |
|---|---|---|---|---|---|---|---|---|---|
| T_{B} (MPa) | 29.12 | 25.40 | 27.43 | 31.31 | 26.00 | 30.00 | 30.68 | 30.20 | 28.33 |
| E_{B} (%) | 570 | 590 | 563 | 520 | 570 | 563 | 567 | 570 | |
| 100% modulus (MPa) | 2.42 | 1.68 | 2.02 | 3.03 | 2.44 | 2.22 | 2.25 | 2.41 | 2.01 |
| 300% modulus (MPa) | 11.34 | 9.64 | 12.78 | 13.21 | 13.26 | 11.82 | 12.31 | 12.73 | 11.32 |

Vulcanization conditions: 160° C., 20 minutes: 100° C., 72 hours heat aging, then blank tension

| | | | | | | | | | |
|---|---|---|---|---|---|---|---|---|---|
| T_{B} (MPa) | 24.15 | 24.82 | 26.61 | 29.96 | 26.31 | 25.69 | 26.49 | 24.72 | 25.12 |
| E_{B} (%) | 407 | 522 | 490 | 461 | 453 | 430 | 447 | 410 | 420 |
| 100% modulus (MPa) | 3.73 | 2.18 | 2.52 | 4.05 | 3.36 | 3.57 | 3.48 | 3.57 | 3.32 |
| 300% modulus (MPa) | 16.97 | 12.53 | 14.94 | 16.24 | 16.88 | 16.29 | 16.57 | 16.87 | 16.83 |

Rate of change after heat aging

| | | | | | | | | | |
|---|---|---|---|---|---|---|---|---|---|
| Rate of change of E_{B} | 71.4 | 88.5 | 87.0 | 79.9 | 87.1 | 75.4 | 79.4 | 71.4 | 73.7 |
| Rate of change of | 149.6 | 130.0 | 116.9 | 122.9 | 127.3 | 137.6 | 134.6 | 144.2 | 148.7 |
| 300% modulus (%) | | | | | | | | | |

Test conditions: 160° C., 60 minutes

| | | | | | | | | | |
|---|---|---|---|---|---|---|---|---|---|
| T5 (min) | 2.88 | 3.73 | 2.42 | 2.52 | 2.39 | 2.51 | 2.40 | 2.54 | 2.58 |
| T95 (min) | 5.43 | 15.65 | 7.58 | 4.82 | 5.96 | 9.34 | 7.48 | 5.07 | 6.78 |
| T-5 (min) | 10.31 | *** | *** | 13.85 | 24.53 | *** | 27.62 | 15.85 | 20.54 |
| Reversion after | 27.0 | 0.2 | 3.6 | 13.9 | 6.5 | 2.6 | 5.2 | 17.5 | 15.8 |
| 60 minutes (%) | | | | | | | | | |

Notes: *1: 5% oil extended powder sulfur, *2: Noccelar NSF (N-t-butyl-2-benzothiazolyl sulfenamide) (made by Ouchi Shinko Chemical), ***: at least 60 minutes.

As explained above, the rubber composition using the vulcanizing agent of the present invention was found to be superior in heat aging resistance and reversion resistance.

While the invention has been described by reference to specific embodiments chosen for purposes of illustration, it should be apparent that numerous modifications could be made thereto by those skilled in the art without departing from the basic concept and scope of the invention.

1. An organic sulfur vulcanizing agent obtained by a reaction between sulfur and norbornene or its derivative.

2. An organic sulfur vulcanizing agent as set forth in claim 1, wherein the organic sulfur vulcanizing agent is expressed by the following formula 1: 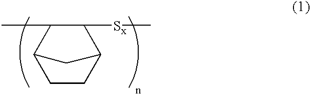
wherein, x is an integer of 1 to 10 and n is an integer of 1 to 200.

3. An organic sulfur vulcanizing agent as set forth in claim 1, wherein the organic sulfur vulcanizing agent is a compound expressed by the following formula 2 or 3:

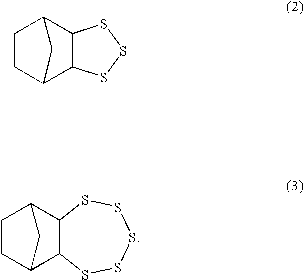

4. An organic sulfur vulcanizing agent obtained by a reaction between sulfur and a dicyclopentadiene in the presence of catalyzing amounts of a vulcanization accelerator and an accelerator activator.

5. An organic sulfur vulcanizing agent as set forth in claim 1, wherein the sulfur is contained in an amount of 30 to 70 wt %.

6. A rubber composition comprised of 0.5 to 10 parts by weight of an organic sulfur vulcanizing agent as set forth in claim 1 or 0.5 to 10 parts by weight of said organic sulfur vulcanizing agent and 0 to 10 parts by weight of sulfur with respect to 100 parts by weight of a diene-based rubber.
